(19) United States
(12) Patent Application Publication
(10) Pub. No.: US 2025/0336393 A1
(43) Pub. Date: Oct. 30, 2025

(54) SYSTEM AND METHOD FOR MOBILE DEVICE BASED REAL-TIME MEDICAL NOTE GENERATION UTILIZING A LOCAL SPEECH RECOGNITION MODEL AND CLOUD BASED LARGE LANGUAGE MODEL

(71) Applicant: Ling Zhou, Mercer Island, WA (US)

(72) Inventor: Ling Zhou, Mercer Island, WA (US)

(21) Appl. No.: 18/646,729

(22) Filed: Apr. 25, 2024

(51) Int. Cl. G10L 15/183; G16H 15/00

(52) U.S. Cl. CPC G10L 15/183; G16H 15/00

(57) ABSTRACT

The present disclosure relates to a system and method for generating medical notes in real-time using a mobile device. The system employs a local, real-time speech recognition model to transcribe medical conversations accurately without relying on cloud-based processing. The transcribed text is then sent to a cloud-based LLM for summarization and generation of a comprehensive medical note. The local speech recognition model is pre-trained on a dataset comprising numerous medical terms and phrases, and it can be iteratively improved through user feedback and additional training. This innovative approach eliminates the ongoing costs associated with cloud-based transcription services while ensuring data privacy and security by keeping sensitive patient information on the mobile device. The system offers the ability to deliver a better product at a price point an entire order of magnitude lower than our competitors.

This application claims the benefit of U.S. Provisional Patent Application No. 63/462,138, filed on Apr. 26, 2023, the contents of which are incorporated herein by reference.

### DESCRIPTION

This invention relates to a novel system and method designed to generate medical notes in real-time. There exist many such automatic medical note generation systems, commonly known as ambient dictation systems. Examples include Microsoft/Nuance DAX or Abridge ambient dictation. These systems employ two main systems to generate the medical note: 1) a transcription system that captures the conversation between the physician and patient and translates it to text and 2) a LLM which then converts the transcript into a standard medical note that can be directly transferred to the electronic medical record (EMR) without further physician intervention. 95% of the cost of these systems lies within the transcription system. We propose and describe a novel approach to transcription that results in an order of magnitude reduction in costs for such systems.

### DETAILED DESCRIPTION

The present disclosure in various embodiments includes a system and method for real-time medical note generation using local speech recognition for transcription and cloud-based large language model (LLM) summarization. The system in some examples includes two main components: A novel, real-time, local speech recognition model capable of running on a mobile device which generates a transcript and a cloud-based LLM for summarization and generation of the note.

Existing medical note generation systems utilize cloud-based, or otherwise off-device, transcription for several reasons. Accurate transcription, especially the ability to recognize medical terms, can be a compute-intensive process in various examples requiring specialized hardware that typically cannot run smoothly on today's mobile devices. Another is simply the difficulty of designing a highly accurate real-time local transcription system from scratch. Finally, many accessible forms of local transcription are not HIPAA compliant, making their use in a medical setting impossible. For example, Apple's iPhone's built-in transcriber can theoretically work on a local basis, but it is explicitly not HIPAA compliant, as Apple uses the voice data for further training of their existing technologies.

One of the most significant drawbacks of cloud-based transcription models is their ongoing cost. Despite some price reductions over the past several years, cloud-based transcription systems typically charge between $1 and $3 per hour of transcription time. For medical dictation, the costs are even higher, ranging from $4 to $5 per hour. Considering that a physician may easily use 3-4 hours of transcription time per day, the monthly cost can quickly add up to over $200 for just a single user. In stark contrast, our transcription system, which runs entirely on a local mobile device, is essentially free to use once deployed.

Even when transcription is routed to a local private server rather than a cloud-based one, the expenses associated with maintaining and operating a server capable of handling simultaneous transcription requests in real-time can be substantial. These costs often exceed those of performing the transcription entirely on the mobile device. Consequently, current automated medical note generation systems that rely on cloud or server-based transcription must charge significantly more than those that utilize local transcription. The cost of cloud-based transcription is much more expensive than similar access to LLMs which perform the conversion of the transcript into medical notes, thus it's much more impactful to localize the processing of the transcription than it is to localize the LLM.

Our innovative approach to local, real-time transcription not only eliminates the ongoing costs associated with cloud-based services but also enhances data privacy and security. By keeping all transcription processing on the mobile device, we ensure that sensitive patient information never leaves the device, greatly reducing the risk of unauthorized access or breaches. This local processing also helps minimize the potential for HIPAA violations, as it reduces the number of functions that depend on the cloud and the associated risks of exposing sensitive patient-identifying information.

Transcription workflow in some embodiments is described: The transcription process is initiated by harnessing the mobile device's inherent recording capabilities to capture dialogues between healthcare providers and patients. Utilizing frameworks like AVFoundation on iOS devices, our system captures audio via the device's built-in microphone and continuously monitors the live audio stream. To optimize real-time transcription, our system employs several additional strategies: 1) Segmented Audio Processing: The system divides ongoing conversations into manageable, discrete audio segments for immediate processing, minimizing delays and potential transcription inaccuracies due to abrupt pauses. 2) Overlapping Audio Segments: To ensure continuity and mitigate risks of mid-word cut-offs, our approach involves slightly overlapping audio segments. While this method demands higher processing power, it significantly enhances transcription accuracy. 3) Asynchronous Parallel Processing: By adopting an asynchronous, parallel processing framework, our system efficiently manages larger audio segments, offering rapid transcription without overwhelming the device's processing capabilities. Following the initial audio capture, the audio is processed through a state-of-the-art neural network that has been extensively trained on a diverse dataset of medical dialogues, including various accents, dialects, and medical terminologies. This neural network leverages deep learning techniques to accurately transcribe the nuanced and technical language of healthcare conversations into text. The transcription model not only recognizes words but also understands context, significantly reducing errors and improving the accuracy of the transcribed text.

A distinguishing feature of the present technology is its inherent mechanism for continuous learning and adaptation. The model is designed to evolve by systematically analyzing corrections and input from healthcare professionals, facilitating the ongoing integration of newly identified medical terminologies and enhancing its proficiency in recognizing user-specific speech nuances. This process of perpetual refinement and personalization ensures that the system remains at the forefront of medical transcription technology, offering an evolving accuracy that is tailored to individual users over time and effectively learning from the medical community it serves.

As of the filing date of this application, the inventor is not aware of any prior art that achieves the same level of accuracy, adaptability, and personalization in an offline, local transcription model for the medical sector. The present invention's unique combination of features and its ability to continuously learn and evolve based on user input distinguish it from existing technologies and establish its novelty and non-obviousness in light of the prior art.

Beyond static training, a distinctive feature of our system is its incorporation of continuous learning and adaptation mechanisms. This innovative approach allows the model to dynamically refine its transcription capabilities based on user interactions and feedback. As healthcare professionals use the system, it collects data on corrected transcriptions, recognizing patterns in errors and integrating new medical terminology that emerges in practice. To our knowledge, no other existing offline, local transcription models demonstrate this level of medical term recognition, adaptability, and personalization at the time of this application.

In various embodiments, (e.g., once some of all of the above process is complete), the transcription of the care provider-patient interaction can be sent via a secure protocol to a cloud based LLM API, (e.g., OpenAI's GPT or Anthropic's Claude), with the prompt to, for example, “convert the transcript into a medical note.” The LLM is able to take natural language as input and produce text-based results with high accuracy. There are infinite variations of the aforementioned prompt, each of which will produce slightly different results, but the vast majority will produce a high quality, ready to use medical note. As of the writing of this patent application, there are many different versions of LLMs with varying degrees of ability. Some embodiments can use GPT 3.5, GPT 4.0, Claude 3, or the like.

In various embodiments, LLMs may one day run offline on local devices as well. The introduction of mobile hardware specifically designed to LLMs are being developed by Apple and other mobile device manufacturers will open the possibility of eliminating cloud based LLMs in the future. However, this will be much less impactful, as the transcription process still represents the brunt of processing power and cost.

Some similar products can be expensive because they aim for seamless integration with existing medical record systems which can allow a generated medical note to be directly inserted into the care giver's EMR. The cost of such integrations can easily exceed 7 figures in some examples, but the time savings afforded by these exercises save mere seconds off each transaction. In contrast, various embodiments discussed herein, such as a method of transferring the note from the mobile device to the device where the care provider's electronic medical record system is located is novel, and can be fast, and secure in various example. Because various embodiments require no integration, some such embodiments can save users millions in upfront cost.

Accordingly, various embodiments include a QR scanning system to provide a method of data transaction between the mobile device and the device which contains the EMR. In one example, once the medical note has been generated, the user can open a secure website through their internet browser which displays a QR code. When the QR code is scanned by the mobile device, the previously generated medical note is sent securely to the server hosting the website, inserting its contents into a SQL table corresponding to the QR code's key. Once the website sees that the SQL table corresponding to its key has been updated, it displays the newly received data on the web browser. There is a convenient button to copy and paste the generated medical note into the EMR, which in various examples can be a quick and easy process.

Further embodiments can include systems that can rival a full integration into the EMR. For example, some embodiments can include a desktop app and web app that are continuously in real-time connection with the mobile device app. This can be achieved via many suitable protocols such as, but not limited to, WebSockets, MQTT, XMPP, Socket.IO, or Firebase Realtime Database. We also considered using secure email, FTP transfer, and other protocols. However, in some examples, a QR code-based approach can be desirable because it can extremely lightweight and portable, requiring absolutely no integration need on the client's end.

The described embodiments are susceptible to various modifications and alternative forms, and specific examples thereof have been shown by way of example in the drawings and are herein described in detail. It should be understood, however, that the described embodiments are not to be limited to the particular forms or methods disclosed, but to the contrary, the present disclosure is to cover all modifications, equivalents, and alternatives. Additionally, elements of a given embodiment should not be construed to be applicable to only that example embodiment and therefore elements of one example embodiment can be applicable to other embodiments. Additionally, in some embodiments, elements that are specifically shown in some embodiments can be explicitly absent from further embodiments. Accordingly, the recitation of an element being present in one example should be construed to support some embodiments where such an element is explicitly absent.

What is claimed is:

1. An automated medical note writing system comprising a mobile device with a local, on-device, real-time speech recognition model capable of accurately transcribing medical conversations without the need for cloud-based processing when used in conjunction with a LLM, thereby significantly reducing the system's overall cost.
